(19) United States
(12) Patent Application Publication
(10) Pub. No.: US 2023/0146285 A1
(43) Pub. Date: May 11, 2023

(54) Wound Disinfection System

(71) Applicant: Farrokh Mohamadi, Irvine, CA (US)

(72) Inventor: Farrokh Mohamadi, Irvine, CA (US)

(21) Appl. No.: 18/046,706

(22) Filed: Oct. 14, 2022

Related U.S. Application Data
(60) Provisional application No. 63/255,743, filed on Oct. 14, 2021.

(51) Int. Cl. A61L 2/00

(52) U.S. Cl. CPC A61L 2/0064

(57) ABSTRACT

A microwave device for a disinfection treatment of a human being is disclosed. The microwave device includes a radiation pad including an array of antennas and a microwave generator configured to excite the array of antennas to cause each antenna in the array of antennas to transmit a continuous-wave microwave signal. Each antenna is arranged in the radiation pad so that as the radiation pad is positioned adjacent a treatment area of the human being the continuous-wave microwave signals from the array of antennas combine in the near field in the treatment area to inactivate microbial pathogens comprising bacteria or fungus.

### CROSS-REFERENCE TO RELATED APPLICATIONS

This application claims the benefit of U.S. Provisional Application No. 63/255,743 filed Oct. 14, 2021, the contents of which are hereby incorporated by reference in their entirety.

### TECHNICAL FIELD

This application relates generally to the disinfection of a treatment area of a human being by inactivating microbial pathogens with microwave energy.

### BACKGROUND

Chronic wounds have an annual prevalence of approximately 1-2% of the Medicare population alone, affecting at least 6.5 million people with annual treatment costs of $25 billion or more. The need for cost-effective wound management modalities has burgeoned due to several factors: the aging population has led to an increase in the overall incidence of chronic diseases, which are associated with chronic wound development, such as diabetes, peripheral vascular disease (venous and arterial), and pressure injuries, as well as an overall rise in the incidence of traumatic and surgical wounds. Other co-morbid conditions, such as cancer and autoimmune disorders contribute to the burden of chronic wound development. Approximately 90% or more of chronic wounds are affected by “bio-film”, which is polymicrobial colonization that is impervious to systemic and most topical antibiotics that contributes to chronicity by promoting a sustained inflammatory state, preventing the normal progression of healing.

Acute wound development is also a growing problem due to an increased incidence of trauma in both the civil and military sectors as well as the number of surgical procedures performed, which is expected to rise over time. Post-operative surgical site infections (“SSIs”) have an enormous impact on patient care and outcomes, costing the U.S. healthcare system billions of dollars annually. For example, SSIs are the most common healthcare-associated infection and result in widespread human suffering and economic loss. Each year, more than 290,000 surgical patients in the United States develop an infection within 30 days of their operation. SSIs also account for an estimated $10 billion in additional healthcare costs and more than 13,000 of those people die. Further, postsurgical infections increase the length of postoperative hospital stays by 7-10 days, as well as rates of hospital re-admission, expenses, and rates of death.

Current techniques to treat wound infections, as well as management of chronic wounds such as affected by bio-film includes the use of strong chemical inactivation, ultraviolet (UV) irradiation, and microwave thermal heating. Unfortunately, all these methods adversely affect healthy tissue. For example, chemical treatments are often poisonous and/or carcinogenic. Similarly, UV irradiation only effects the surface of an object and does not penetrate sufficiently within infected tissue. In addition, microwave thermal heating techniques generally require power levels that may be dangerous or unhealthy. As such, there is a need for an improved system and method for destroying microbial pathogens that is safe to utilize.

### SUMMARY

A microwave device for a disinfection treatment of a human being is disclosed. The microwave device includes a radiation pad including an array of antennas and a microwave generator configured to excite the array of antennas to cause each antenna in the array of antennas to transmit a continuous-wave microwave signal. Each antenna is arranged in the radiation pad so that as the radiation pad is positioned adjacent to a treatment area of the human being the continuous-wave microwave signals from the array of antennas combine in the near field in the treatment area to inactivate microbial pathogens comprising bacteria or fungus.

In an example of operation, a microwave device performs a method for disinfection. The method includes positioning a radiation pad including an array of antennas adjacent a treatment area of a human being to cause the array of antennas to transmit a plurality of continuous-wave microwave signals that combine in the near field to form a combined signal in the treatment area. The combined signal inactivates microbial pathogens in the treatment area. The microbial pathogens comprise bacteria or fungi.

Other devices, apparatuses, systems, methods, features, and advantages of the invention will be or will become apparent to one with skill in the art upon examination of the following figures and detailed description. It is intended that all such additional devices, apparatuses, systems, methods, features, and advantages be included within this description, be within the scope of the invention, and be protected by the accompanying claims.

### BRIEF DESCRIPTION OF THE DRAWINGS

The invention may be better understood by referring to the following figures. The components in the figures are not necessarily to scale, emphasis instead being placed upon illustrating the principles of the invention. In the figures, like reference numerals designate corresponding parts throughout the different views.

FIG. 1 is a system block diagram of an example implementation of a microwave device for inactivation of microbial pathogens in accordance with the present disclosure.

FIG. 2 illustrates a hardware setup of an example implementation of the microwave device shown in FIG. 1 in accordance with the present disclosure.

FIG. 3 is an illustration of an example application of the microwave device shown in FIG. 1 in accordance with the present disclosure.

FIG. 4 illustrates a top view of a radiation pad that includes an antenna array and a perspective view of a portion of the antenna array in accordance with the present disclosure.

FIG. 5 illustrates perspective views of the radiation pad that includes an antenna array in flat and bended forms in accordance with the present disclosure.

FIG. 6 illustrates a top view of a radiation pad integrated with sensors in accordance with the present disclosure.

FIG. 7 illustrates time captured radiation patterns of the antenna array in a radiation pad in accordance with the present disclosure.

FIG. 8 illustrates a near-field strength of microwave energy transmitted from the antenna array in accordance with the present disclosure.

FIG. 9 illustrates agnostic microbial pathogen count reduction versus dwelling time of exposure in accordance with the present disclosure.

FIG. 10 illustrates the effectiveness of inactivating fungus by using the microwave device shown in FIG. 1 in accordance with the present disclosure.

FIG. 11 is a flowchart of an example of an implementation of a method performed by the microwave device shown in FIG. 1 in accordance with the present disclosure.

### DETAILED DESCRIPTION

Microwaves have been used to inactivate viruses. With regard to this inactivation, it has been proposed that a “spring resonance” occurs with regard to vibration of the virus itself. Under such a hypothesis, the microwave radiation frequency is chosen based upon a spring resonance frequency established by the virus diameter. If such a hypothesis were correct, then the microwave frequency for inactivation of microorganisms such as bacteria and fungi would require a dramatically different frequency. But it was discovered herein that a similar or even identical microwave frequency inactivates bacteria and fungus. Because this inactivation cannot occur under a spring resonance theory, it is hypothesized herein that the inactivation occurs due to a microwave-induced heating of water molecules surrounding and within the microorganism.

Advantageously, it is discovered herein that bacteria and fungi may be inactivated using relatively low-power microwave energy such as at a power level of approximately 10 Watts. To achieve this advantageous result, a treatment pad is disclosed that includes an array of antennas. For low-cost operation, a continuous-wave generator generates a continuous-wave microwave generator signal to excite the antennas. In some embodiments, an antenna-to-antenna spacing between adjacent antennas in the array may be less than one-half of a wavelength of the continuous-wave microwave generator signal. The continuous-wave microwave generator signal from the generator is distributed substantially equally to each antenna. Each antenna thus transmits a microwave signal that is a fraction of the continuous-wave microwave generator signal. Advantageously, the spacing of the antennas assists in a near-field combination of the antenna-transmitted signals within a treatment area of a human being. In this fashion, the array distributes the transmitted signals so that a safe power level is achieved in the treatment area, yet enough power is provided to inactivate microorganisms without causing pain or harm to the patient.

This approach disclosed herein stands in sharp contrast to previous attempts to inactivate bacteria using microwaves. For example, pulsed microwave radiation has been used on some gram-positive bacteria, such as S. aureus and E. coli cultures, which were exposed to pulsed microwave radiation (e.g., pulse duration of 60 ns, peak frequency of 3.5 GHz) with power density of 17 kW/cm at the free space from samples as an effect to induce electric field of 8.0 kV/cm inside the solution of a falcon tube. Scanning electron microscopy has revealed surface damage in bacterial strains after PMR exposure. The bacterial inactivation by pulsed microwave radiation is attributed to the deactivation of oxidation-regulating genes and DNA damage. However, a power density of 17 kW/cm applied in the pulsed microwave radiation techniques can be dangerous to humans.

Recent research on the inactivation of the Murine coronavirus (“MHV”), a species of coronavirus that infects mice, has shown that microwave-based disinfection technologies have some inactivation effects on viruses. As noted earlier, it was hypothesized that the microwave energy induces self resonance of the virus to fracture the virus. Further research shows that the influenza A (“H3N2”) variant virus was also believed to resonate in a confined-acoustic dipolar mode with microwaves of the same frequency so as to be inactivated. However, viruses are generally orders of magnitude smaller than microbial pathogens. The frequencies of confined-acoustic dipolar modes between a virus and a microbial pathogen are also dramatically different. The existing microwave-based disinfection technologies applied on viruses thus cannot be directly applied to inactivation of microbial pathogens.

A microwave device for contactless disinfection treatment transfers energy not directly to microbial pathogens, but rather to the environment (medium) that surrounds the bacteria or fungus and causes the resonance to occur. At a certain dwelling time, if the vibration frequency of the transferred heat energy to the medium is sufficient, then the capsids crack. Experiments on Staphylococcus aureus (“S. aureus”) presented in this proposal validate the effectiveness of the proposed mechanism for inactivation of microbial pathogens.

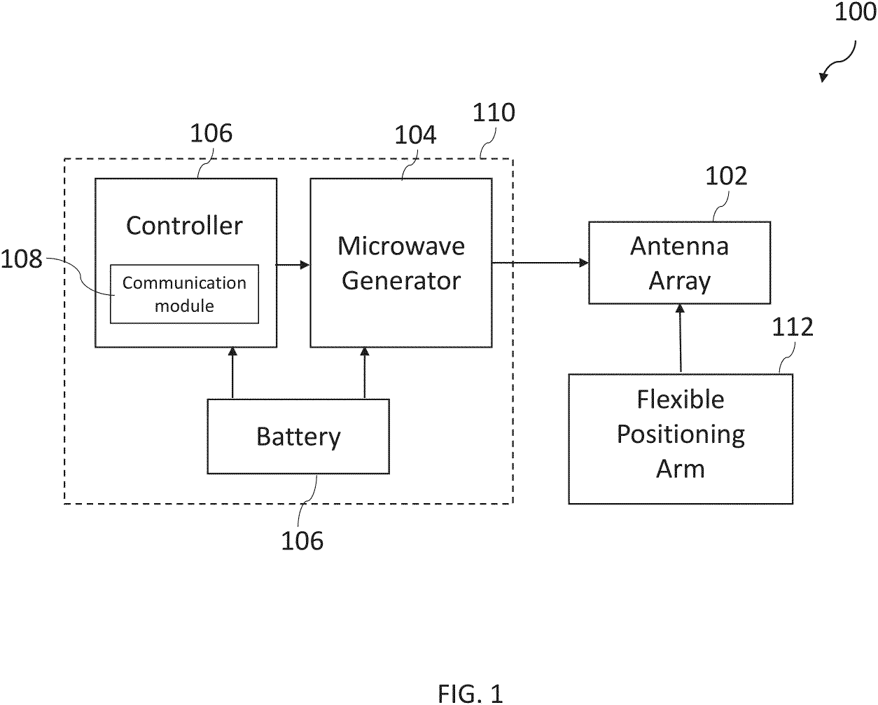

Turning now to the drawings, a system block diagram is shown in FIG. 1 of an example microwave device 100 for inactivation of microbial pathogens in accordance with the present disclosure. In this example, the microwave device 100 includes a radiation pad 102 housing an antenna array, a portable microwave generator 104 generating and providing microwave energy in a continuous wave to the antenna array, an optional controller 106 for control of power level adjustment, frequency selection and/or tuning), and a battery pack 106 powering the controller 106 and the microwave generator 104.

The controller 106 may include a communication module 108 providing Bluetooth and/or WiFi connectivity. The microwave device 100 may also integrate customized solutions with various sensors such as temperature, voltage, current, humidity, pressure, inertial management unit (“IMU”), miniature infrared (“IR”) illuminator, micro camera, and other suitable sensors. For example, various sensors may be integrated in the radiation pad 102. Further, the microwave generator 104, the controller 106, and the battery pack 106 may be included within one integrated module 110.

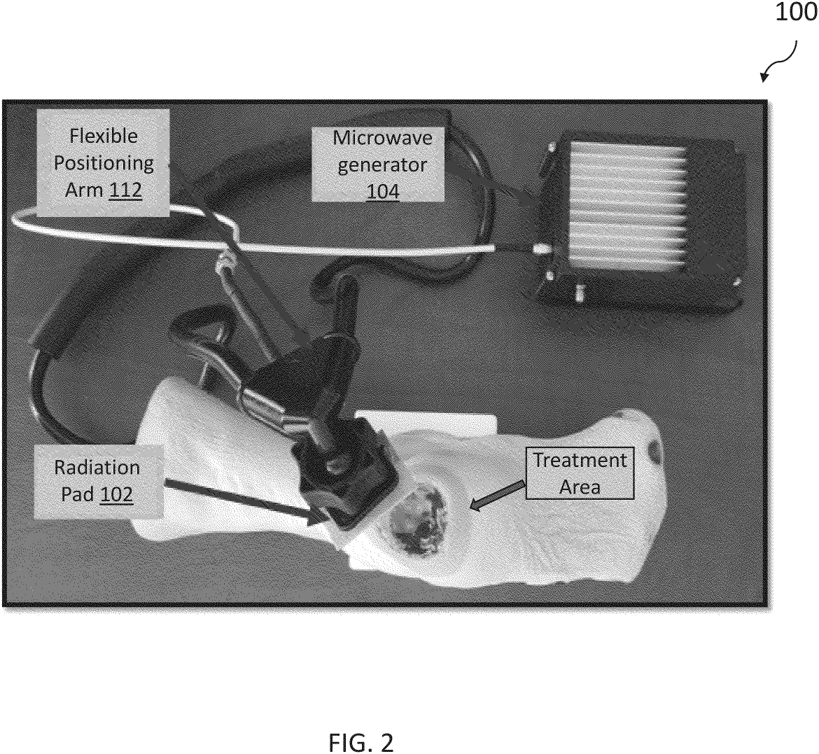

The microwave device 100 may also include a flexible positioning arm 112. The flexible positioning arm 112 is attached to the radiation pad 102 to facilitate securing the radiation pad 102 adjacent to a disinfection treatment area, such as a wound. FIG. 2 illustrates an example implementation of the microwave device 100 in accordance with the present disclosure. The dimensions of the radiation pad 102 is scalable. Although disinfection advantageously may be affected without contacting the radiation pad 102 to a patient, radiation pad 102 may be directly applied to the skin or wound of a patient. In one example, the radiation pad 102 can be used for smaller wounds and measures 2″ × 2″ × 0.5″. For larger wounds, the radiation pad 102 measures 4″ × 4″ × 0.5″, and additional versions allow for even larger sizes, such as 16″ × 16″ × 0.5″, if needed. The system design conveniently facilitates multiple office visits or in-house treatment. In one instance, the portable microwave generator 104 measures 5″ × 5″ × 1″, while a more complex smart microwave generator with connectivity to a cellphone and exposure management software capability may measure 11.5″ × 6″ × 2″. A quick charge battery may be included for 8 hours of continuous operation in one charge. However, since exposures may be limited to 4 minutes in some applications, the system could operate for several months on a single charge. The low-cost rechargeable battery can also be replaced or attached to a charger for on-demand or prolonged operation. The coupling between the radiation pad 102 and the microwave generator 104 may be a durable flexible wiring system that is easily plugged in or decoupled. The quick and easy process to establish connection allows the radiation pad 102 to be easily replaced in case of physical damage or swapped for a pad of more suitable size. The radiation pad 102 can also be reused. In one instance, the modular designs also allow the system to integrate with a library of medications incorporating exchangeable cartridges, microneedles, micropumps, catheters, gels, etc. The system design may have a manual fail-safe backup option for motorized or automated designs.

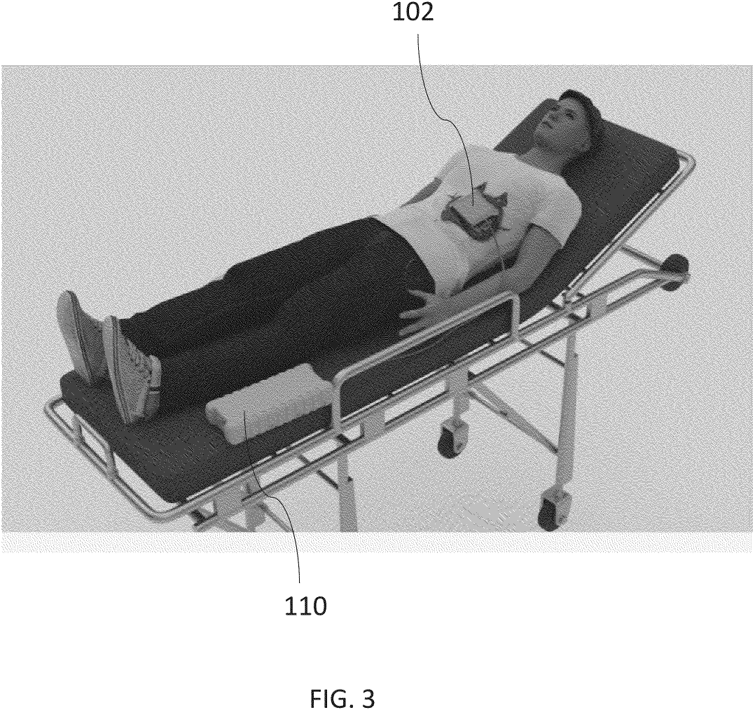

FIG. 3 illustrates an application of the microwave device 100. The radio frequency penetrates bandages and thin layers of liquids; therefore, it can be applied to dressed as well as exposed wounds. In some implementations, the continuous-wave microwave signal that excites the array may be tunable for various frequencies. Various sizes of bacteria have specific harmonic characteristics and microwave radiation may be fine-tuned to a range of frequencies to inactivate them.

In the illustrated application of the microwave device 100, the radiation pad 102 covers a treatment area of a human being. The treatment area can be wound or other infected area such as a nail bed. Adhesives may be used to attach the radiation pad 102 to the treatment area. The integrated module 110, which integrates the microwave generator 104, the battery pack 106, and optionally the controller 106 and peripheral sensors previously mentioned, excites the antenna array in the radiation pad 102 to inactivate microbial pathogens including bacteria or fungus. The system can be combined with other treatments, such as analgesics for pain management, as the system is non-intrusive and has no interference with any other treatment such as the use of oral medication or the intravenous injection of drugs. There are thus no chemical reactions with any pharmacologic molecules from the microwave treatment. Some example antenna array configurations will now be discussed.

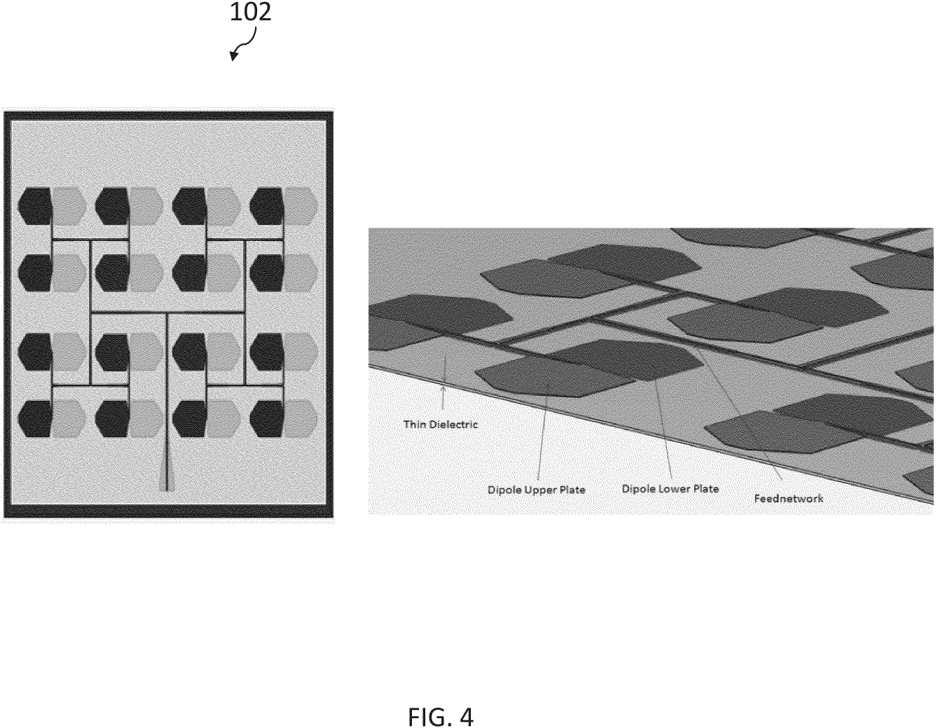

FIG. 4 illustrates an antenna array for integration with the radiation pad 102. In this example, a 4 × 4 array of antennas operating between 4 GHz to 12 GHz is illustrated. In some embodiments, the 4 × 4 array may operate between 6 GHz to 10 GHz. As discussed above, depending on the treatment area of a human being, the antenna array size may be varied, such as an 8 × 8 array of antennas, a 16 × 16 array of antennas, or a 32 × 32 array of antennas. Each of the antennas is fed by a feed network. Each antenna is a dipole although other types of antennas such as patch antennas may also be implemented. The dipole array may be integrated with a low dielectric (foam type) material and coated with perylene for insulation from water. A thin coating does not impact the performance of the microwave exposure. As shown in FIG. 4, each dipole may include a dipole upper plate that is formed on one surface of the low-dielectric film and a dipole lower plate that is formed on an opposing side of the low-dielectric film. An advantage of the antenna array is ease of manufacturing and capability to be scaled and produced in large volumes.

Since the combination of the transmitted signals from each antenna occurs in the near field in the treatment area, the antenna spacing for the array need not be one-half the wavelength of the transmitted microwave signals but instead may be smaller. For example, in one implementation, the antenna spacing between neighboring ones of the antennas in the antenna array may be 0.4 times the wavelength of the microwave signal frequency. In this fashion, the spacing of the antenna array advantageously aids in the near field combination of the transmitted signals in the treatment area. The results are quite advantageous with respect to inactivating bacteria and fungi without causing harm to human tissue.

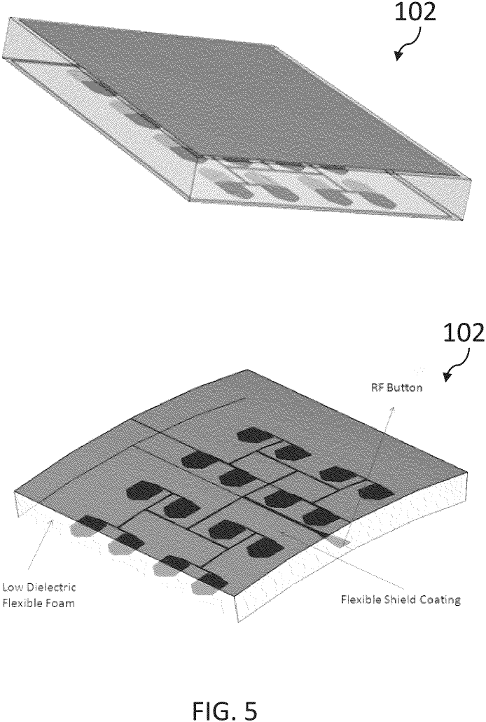

Due to the flexibility of the dielectric layer or film, the radiation pad may be conformal to the human torso. A conformal antenna layer is shown in FIG. 5, where each dipole is printed on a very thin layer (e.g., 100 microns) low dielectric constant polymer sheet. The polymer sheet is then encapsulated in flexible low dielectric constant foam and a shielding layer of thin copper plate is attached to it. The thin copper layer directs the microwaves to the treatment area for more effective disinfection. The entire assembly may then be coated with perylene to isolate the unit from the environment.

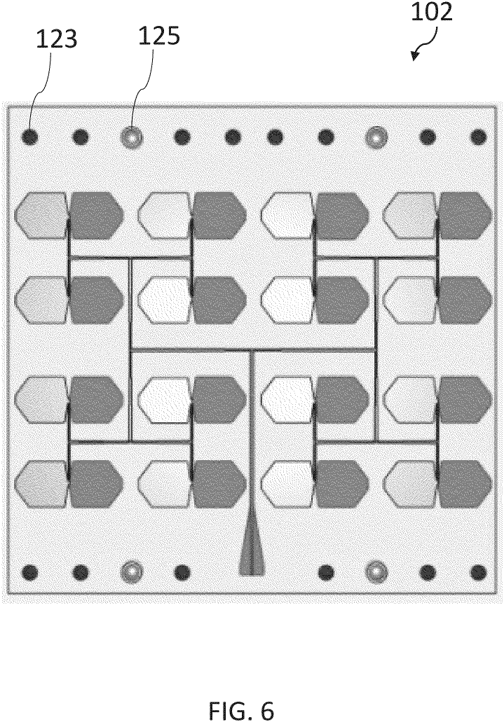

FIG. 6 illustrates that other miniature sensors, such as temperature, pressure, humidity, micro camera, or a light emitting diode (“LED”) can be integrated for the radiation pad 102 to provide adaptive signal processing capability to monitor the wound disinfection and manage heat treatment. In the illustrated instance, the radiation pad 120 is integrated with an array of micro light emitted diodes (“LEDs”) 123 and four charge coupled (“CCD”) micro cameras 125 for in-situ heat based wound treatment and healing observation.

The microwave generator 104 is configured to excite the array of antennas to cause each antenna in the array of antennas to transmit a continuous-wave microwave signal. Each antenna is arranged in the radiation pad 102 so that as the radiation pad 102 is positioned adjacent a treatment area of the human being the continuous-wave microwave signals from the array of antennas combine in the near field in the treatment area to inactivate microbial pathogens comprising bacteria or fungus.

With regard to the spring resonance theory, the influenza A (“H3N2”) variant virus is believed to resonate in a confined-acoustic dipolar mode so as to inactivated. It is hypothesized herein that the energy transfer from the microwave signal is not delivered directly to the viruses, but rather to the environment such as water molecules that surround the viruses and causes the resonance to occur. At a certain exposure time (dwelling time), if the vibration frequency of the transferred energy to the medium equals the spring resonance energy, then the viruses inactivate. Based on recent in-vitro tests, a similar model is proposed herein to inactivate larger microbial pathogens.

From transmission electron microscope images, it is known that the S. aureus is basically a spherical ball with packed genomes inside. Since the protein and genome have similar mechanical properties, the S. aureus can be treated as a homogenous sphere for the estimation of dipolar vibration frequencies. The bacteria’s average size is about 1 µm. The Drude-Lornze model of the spring force can be re-written with the force exerted from the electrical field as shown in Equation 1:

$$m \frac{{d}^{2} z}{d {t}^{2}} + k z + b \frac{d z}{d t} = q E$$

$$\text{E} \text{=} \text{E0} \text{cos} \left(ω \text{t}\right)$$

$$\text{z} \left(\text{t}\right) = \text{A cos} \left(ω \text{t -} θ\right)$$

$$b = \frac{{ω}_{0} m}{Q}$$

where:
b= damping coefficient
k= effective spring constant
m= reduced mass
Q= resonation quality factor
q= total amount of charge distributed in the core and shell region of a virus or microbe
E= applied electric field
z(t)= forced displacement
A= amplitude of the forced displacement
θ= phase delay between the displacement and the applied electric field

For a synchronous oscillation of the bacteria’s mass as a result of being exposed to the electrical field, Equations 2, 3, and 4 can be solved with the shown parameters. By solving resonance Equation 1 with synchronous vibrations caused by the exposed electric field, we can obtain a threshold power A to inactivate the bacteria:

$$A = \frac{q {E}_{0}}{m \sqrt{{\left({ω}_{0} {}^{2} - {ω}^{2}\right)}^{2} + {\left(\frac{{ω}_{0} ω}{Q}\right)}^{2}}}$$

$$S = \frac{1}{2} {ε}_{r} {ε}_{0} c {E}^{2}$$

where:
E₀= absorption electric field
S= Intensity flux
εᵣ= relative permittivity of virus or microbe
ε₀= free space permittivity
c= speed of light in free space
ω₀= resonance angular frequency

Alternatively, the impact of the intensity flux for the magnetic field can be written as:

$$S = \frac{{E}^{2}}{2 {μ}_{0} c}$$

where:
E= absorption magnetic filed
S= Intensity flux
µ₀ = free space permeability
C = speed of light in free space

The reported values of E= 80 V/m to inactivate S. aureus and E. coli by 4 and 6 log, respectively, can be used to address the safety of the microwave exposure. Based on the Institute of Electrical and Electronics Engineers (“IEEE”) Microwave Safety Standard, the spatial averaged value of the power density (“SAVPD”) in the air or in open public spaces shall not exceed the equivalent power density of 100(f/3)^⅕ W/m at frequencies between 3 and 96 GHz, (f is in GHz). Accordingly, the SAVPD for the proposed microwave device corresponds to 115 W/m at 6 GHz, 122 W/m at 8 GHz, and 127 W/m at 10 GHz for averaged values of the power densities in the air. Assuming all of the microwave power in the air is 100% transmitted into a specimen, and by taking the dielectric constant of water of 71.92 (6 GHz), 67.4 (8 GHz), and 63.04 (10 GHz) for calculations, these fields then correspond to the average electric field magnitude of 101 V/m (6 GHz), 106 V/m (8 GHz), and 110 V/m (10 GHz) inside the water-based specimens. Hence, the required threshold of electric field magnitudes at the resonant frequency (80 V/m) to destroy the bacterial structures is within the IEEE Microwave Safety Standard at 6 to 10 GHz (101 to 110 V/m), indicating a high structure-resonant energy transfer efficiency.

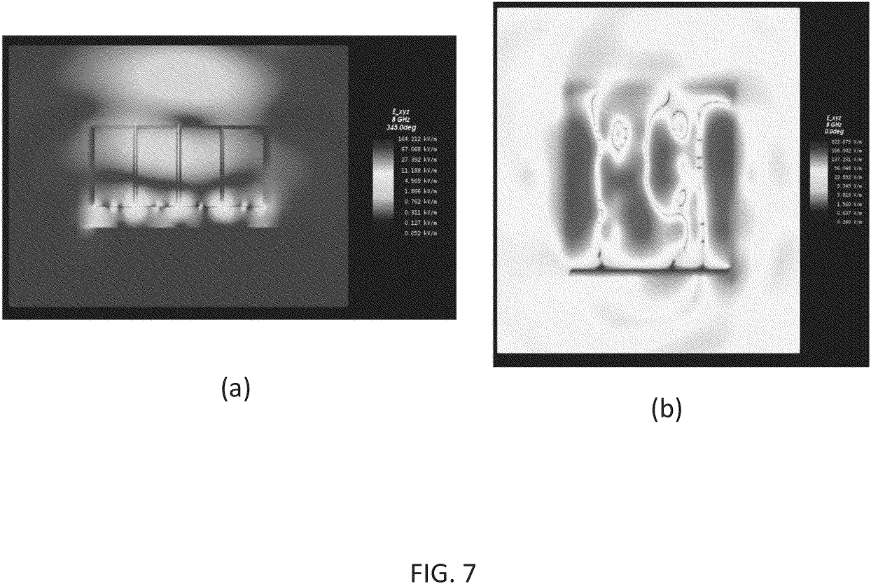

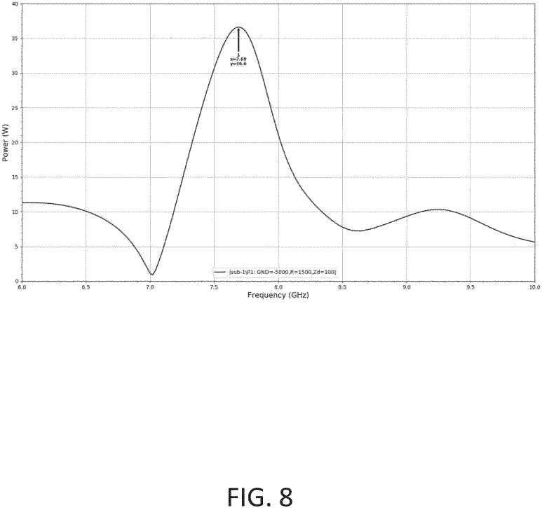

FIG. 7(a) depicts simulation results of the electric field through the antenna array vertically towards a treatment area. FIG. 7(b) illustrates the time snapshot of uniformity of the electric field within the treatment area. As seen from these simulation images, the near-field strength is about 1866 V/m (FIG. 7(b)) for a Gaussian transmitted power of 36.6 W as shown in FIG. 8. The nominal power transmitted from the radiation pad 102 will be about 10 W nominal (7.8 W actual), accordingly, the electric field will be lower by a factor of (36.6/7.8)² = 21.9 than the simulation value, hence, the exposure will be about 1866/21.9= 85 V/m, which is slightly higher than the 80 V/m to fracture the microbial structure.

In some instances, the microwave generator 104 is configured to excite the array of antennas with a power of 7 to 11 Watts. The combined signal that forms in the near field in the treatment area may have an energy in a range from 80 V/m to 110 V/m so as to fracture the microbial pathogens by inducing a resonance in a medium surrounding the microbial pathogens.

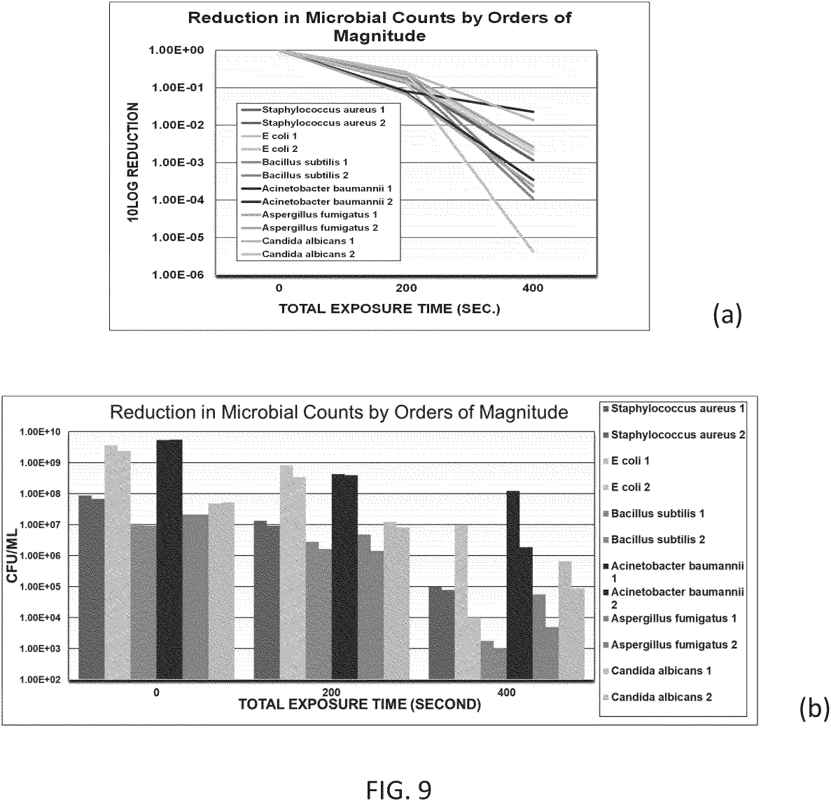

Referring to FIG. 9, the effectiveness of the treatment to inactivate various bacteria which may infect surface and deep wounds is illustrated. Particularly, FIG. 9(a) shows a 10 Log reduction graph based on agnostic pathogen microbial count reduction results on glass; FIG. 9(b) shows a pre- and post-exposure graph based on agnostic pathogen microbial count reduction results on glass. Glasses inoculated with the Staphylococcus aureus, Escherichia coli, Bacillus subtilis, Acinetobacter baumannii, Candida albicans, and Aspergillus fumigatus were exposed under the microwave energy carried by a continuous wave. The bacteria were taken from pure stock cultures and grown separately onto Tryptone Soya Agar (“TSA”) and incubated at 35±1° C. for 24 hours. All cultures were used to harvest colonies which were suspended into 10 mL Trypticase Soy Broth (“TSB”). The cell concentration of 10⁸ CFU/ml for S. aureus, 2 × 10⁹ CFU/ml for E. coli, and 10⁷ CFU/ml were used for the microwave exposures. The glasses with the cell suspensions of 100 uL were placed individually under the radiation pad 102 and exposed to microwaves at a nominal power of 10 W. Increments of 10 MHz frequency sweeps with variable exposure times at each frequency (referred to as the dwelling time) were used for each test. The exposure time and dwelling time were manually set on the laptop. The temperature changes in the suspensions were monitored with a Berrcom Non-Contact Infrared Thermometer (JXB-178). After the treatments, the glasses were vortexed in tubes, serially diluted, and plated for growth on TSA. Plates were incubated for 24-48 hours at 35±1° C.

Since the only common material between the virus and microbe is the lipid layer, it can be postulated that the process is breaking the capsids and neither impacting the DNA (microbe) nor the RNA (virus). This important finding of not impacting the RNA and DNA is critical for the wound’s healing process as the human DNA is not affected. The choice of higher frequency is also ideal for implementation of smaller pad size. In some instances, the operational frequency of the continuous wave can be selected from a list of frequencies pre-stored in the microwave generator 104 or fine-tuned by the microwave generator 104. The operational frequency is specifically targeted on a type of the microbial pathogen. The operational frequency may be between 4 GHz to 12 GHz. In furtherance of instance, the operational frequency may be between 6 GHz to 10 GHz.

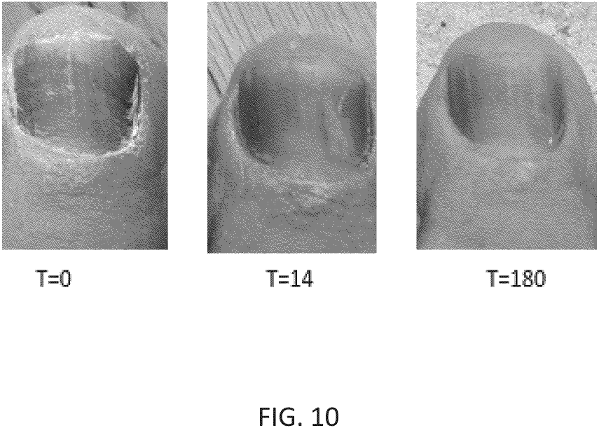

Referring to FIG. 10, the effectiveness of the treatment to inactivate fungus within a nail bed is illustrated. A toenail from a volunteer was exposed for 4 minutes twice in two weeks intervals and monitored over a period of time for effectiveness. As a result of this test, the microwave device disclosed herein may also be capable of treating distal subungual onychomycosis, which is thought to be secondary to sub-acute infection of the keratin in the nail matrix by the Trichophyton fungus.

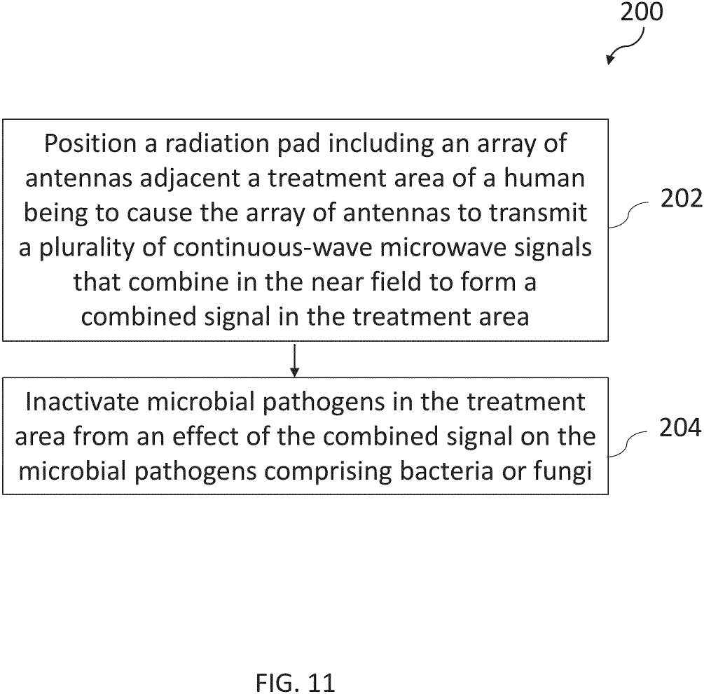

In FIG. 11, a flowchart is shown of an example of an implementation of method 200 for inactivation of microbial pathogens with a microwave device 100. The method 200 starts at operation 202 by positioning a radiation pad including an array of antennas adjacent a treatment area of a human being to cause the array of antennas to transmit a plurality of continuous-wave microwave signals that combine in the near field to form a combined signal in the treatment area. The method 200 proceed to operation 204 to inactivate microbial pathogens in the treatment area from an effect of the combined signal on the microbial pathogens comprising bacteria or fungi.

It will be understood that various aspects or details of the disclosure may be changed without departing from the scope of the disclosure. It is not exhaustive and does not limit the claimed disclosures to the precise form disclosed. Furthermore, the foregoing description is for the purpose of illustration only, and not for the purpose of limitation. Modifications and variations are possible in light of the above description or may be acquired from practicing the disclosure. The claims and their equivalents define the scope of the disclosure. Moreover, although the techniques have been described in language specific to structural features and/or methodological acts, it is to be understood that the appended claims are not necessarily limited to the features or acts described. Rather, the features and acts are described as example implementations of such techniques.

It will also be understood that various aspects or details of the invention may be changed without departing from the scope of the invention. It is not exhaustive and does not limit the claimed inventions to the precise form disclosed. Furthermore, the foregoing description is for the purpose of illustration only, and not for the purpose of limitation. Modifications and variations are possible in light of the above description or may be acquired from practicing the invention. The claims and their equivalents define the scope of the invention.

The description of the different examples of implementations has been presented for purposes of illustration and description, and is not intended to be exhaustive or limited to the examples in the form disclosed. Many modifications and variations will be apparent to those of ordinary skill in the art. Further, different examples of implementations may provide different features as compared to other desirable examples. The example, or examples, selected are chosen and described in order to best explain the principles of the examples, the practical application, and to enable others of ordinary skill in the art to understand the disclosure for various examples with various modifications as are suited to the particular use contemplated.

What is claimed is:

1. A microwave device for a disinfection treatment of a human being, comprising:
a radiation pad including an array of antennas; and
a microwave generator configured to excite the array of antennas to cause each antenna in the array of antennas to transmit a continuous-wave microwave signal, wherein each antenna is arranged in the radiation pad so that as the radiation pad is positioned adjacent a treatment area of the human being the continuous-wave microwave signals from the array of antennas combine in the near field in the treatment area to inactivate microbial pathogens comprising bacteria or fungus.

2. The microwave device of claim 1, wherein the microbial pathogens include at least one of staphylococcus aureus, escherichia coli, bacillus subtilis, acinetobacter baumannii, candida albicans, or aspergillus fumigatus.

3. The microwave device of claim 1, wherein the continuous-wave microwave signals from the array of antenna that combine in the near field in the treatment area are configured to induce a resonance of water molecules that surround the microbial pathogens to fracture a membrane of the microbial pathogens.

4. The microwave device of claim 1, wherein a spacing between adjacent ones of the antennas in the array of antennas is less than one-half of a wavelength of a frequency of each continuous-wave microwave signal.

5. The microwave device of claim 4, wherein the spacing is about 0.4 times the wavelength of the frequency of each continuous-wave microwave signal.

6. The microwave device of claim 1, wherein each antenna in the array of antennas comprises a dipole antenna.

7. The microwave device of claim 6, wherein radiation pad includes a flexible dielectric substrate, and wherein each dipole antenna includes an upper plate disposed on an upper surface of the flexible dielectric substrate and a lower plate disposed on a lower surface of the flexible dielectric substrate.

8. The microwave device of claim 1, wherein each antenna in the array of antennas comprises a patch antenna.

9. The microwave device of claim 1, wherein the array of antennas is one of a 4 × 4 array of antennas, an 8 × 8 array of antennas, a 16 × 16 array of antennas, or a 32 × 32 array of antennas.

10. The microwave device of claim 1, wherein the portable microwave generator is operable to tune a frequency of the continuous-wave microwave signals.

11. The microwave device of claim 1, wherein the microwave generator is configured to excite the array of antennas with a power of 7 to 11 Watts.

12. The microwave device of claim 1, further comprising:
a battery pack configured to power the microwave generator.

13. The microwave device of claim 6, further comprising a feed network in the radiation pad configured to couple the microwave generator to the array of antennas.

14. The microwave device of claim 10, wherein the microwave generator is operable to tune the continuous frequency in a band within 4 GHz to 12 GHz.

15. The microwave device of claim 1, wherein the treatment area comprises a wound in the human being.

16. The microwave device of claim 1, wherein the treatment area comprises a nail bed of the human being.

17. A method for disinfection with a microwave device, comprising:
positioning a radiation pad including an array of antennas adjacent a treatment area of a human being to cause the array of antennas to transmit a plurality of continuous-wave microwave signals that combine in the near field to form a combined signal in the treatment area; and
inactivating microbial pathogens in the treatment area from an effect of the combined signal on the microbial pathogens, wherein the microbial pathogens comprise bacteria or fungi.

18. The method of claim 17, wherein a frequency of the continuous-wave microwave signals is within a frequency band from 4 GHz to 12 GHz.

19. The method of claim 18, wherein the combined signal has an energy in a range from 80 V/m to 110 V/m so as to fracture the microbial pathogens by inducing a resonance in a medium surrounding the microbial pathogens.

20. The method of claim 17, wherein the treatment area comprises a wound of the human being or a nail bed.
